(19) United States
(12) Patent Application Publication
(10) Pub. No.: US 2023/0069345 A1
(43) Pub. Date: Mar. 2, 2023

(54) MICROBIOME DELIVERY PLATFORM

(71) Applicant: Augustine T. Zvinavashe, Cambridge, MA (US)

(72) Inventor: Augustine T. Zvinavashe, Cambridge, MA (US)

(21) Appl. No.: 17/894,913

(22) Filed: Aug. 24, 2022

Related U.S. Application Data
(60) Provisional application No. 63/236,857, filed on Aug. 25, 2021.

(51) Int. Cl. A61K 9/48; A61K 9/28; A61K 35/741; A61K 9/19; A61K 47/42; A61K 47/26

(52) U.S. Cl. CPC A61K 9/4891; A61K 9/2873; A61K 35/741; A61K 9/19; A61K 47/42; A61K 47/26

(57) ABSTRACT

The present disclosure generally relates to preservation and delivery of a bioactive composition to a biological subject. In some embodiments, the bioactive composition include bioactives of at least one of microbes, cells, tissue, vaccines, probiotics, antibiotics, vitamins, or mRNA. Some embodiments include preserving the bioactive composition using vitrification of the bioactive composition in a vitrifying mixture of intrinsically disordered proteins and saccharides. The vitrified bioactive composition is configured to release the bioactive composition from the vitrifying mixture. Some embodiments include administering the vitrified bioactive composition to the biological subject, such that the administering includes applying the vitrified bioactive composition to a microbiome of the biological subject. Some embodiments may further include providing an enteric encapsulation of the vitrified bioactive composition that is configured to protect the vitrified bioactive composition from a first set of environmental conditions, but release the vitrified bioactive composition in a second set of environmental conditions.

### PRIORITY

This patent application claims priority from provisional U.S. patent application No. 63/236,857, filed Aug. 25, 2021, entitled, “MICROBIOME DELIVERY PLATFORM,” and naming Augustine Zvinavashe as inventor, the disclosure of which is incorporated herein, in its entirety, by reference.

### FIELD

Illustrative embodiments of the invention generally relate to preserving bioactive components and, more particularly, various embodiments of the invention relate to preserving the bioactive components and delivering the preserved bioactives to biological subjects.

### BACKGROUND

The use of herbs, leaves, and certain animal products for treating people, animals, and plants has been known to back to antiquity, and the of use fresh biological products in modern health and wellness is growing.

A challenge to the use of certain biological products is keeping the product alive during the process of taking the fresh substance and turning it into a commercial product. That is, the biological properties that make the product beneficial for treatment, are often destroyed in the process of taking from the living state to a form that can be included in a product.

### SUMMARY OF VARIOUS EMBODIMENTS

In accordance with one embodiment of the invention, a method of delivery of a bioactive composition to a biological subject includes providing a bioactive composition. The bioactive composition includes bioactives of at least one of microbes, cells, tissue, vaccines, probiotics, antibiotics, vitamins, peptides, RNAi, or mRNA. The method also includes preserving the bioactive composition. The preserving includes vitrification of the bioactive composition in a vitrifying mixture of intrinsically disordered proteins and saccharides to produce a vitrified bioactive composition. The vitrified bioactive composition is configured to release the bioactive composition from the vitrifying mixture. The method also includes administering the vitrified bioactive composition to the biological subject, such that the administering includes applying the vitrified bioactive composition to a microbiome of the biological subject.

The method may further include providing an enteric encapsulation of the vitrified bioactive composition. The enteric encapsulation is configured to protect the vitrified bioactive composition from a first set of environmental conditions. The enteric encapsulation is also configured to breakdown in a second set of environmental conditions, thereby releasing the vitrified bioactive composition.

The enteric encapsulation may include an enteric polymer capsule in which the vitrified bioactive composition is placed. The enteric encapsulation may include an enteric coating that entirely covers the vitrified bioactive composition.

The intrinsically disordered proteins may be at least one of a tardigrade intrinsically disordered protein, a silk fibroin, or regions of tropoelastin, and the saccharides may be a disaccharide. The silk fibroin may be extracted from Bombyx Mori. The disaccharide may be cellobiose, lactose, or trehalose.

The microbes may include at least one of a fungus, a eubacterium, a eukaryotic organism, or an archaebacteria.

The bioactive composition may be one of: anaerobic, aerobic, spore-forming, vegetative, native to a human microbiome, native to an animal microbiome, non-native to the human microbiome, non-native to the animal microbiome, an engineered version of the bioactive composition that is non-native to the human microbiome, or an engineered version of the bioactive composition that is non-native to the animal microbiome. The bioactive composition may be enhanced by the addition of metabolites and/or preservatives.

The biological subject may be a human being. The applying may include applying the vitrified bioactive composition to at least one of skin, a vagina, a penis, a foot, or an armpit of the human being. The applying may include ingesting an enterically encapsulated vitrified bioactive composition in the form of at least one of an oral ingestible pill, an oral ingestible tablet, an ingestible tablet, or a solution.

The vitrified bioactive composition may be ingested in the form of an enterically encapsulated vitrified bioactive composition that enters a digestive track of the biological subject. The enterically encapsulated vitrified bioactive composition may pass through the digestive track intact until the enterically encapsulated vitrified bioactive composition experiences a pH of greater than about 6. The enterically encapsulated vitrified bioactive composition may break down and release the vitrified bioactive composition when it experiences the pH of greater than about 6. The vitrified bioactive composition may release the bioactive composition.

The vitrifying the bioactive composition may lower the metabolism of the bioactives of the bioactive composition, thereby putting the bioactives in stasis. The vitrifying the bioactive composition may protect the bioactive composition from dying due to exposure from oxygen. The vitrified bioactive composition may not be released from the enterically encapsulated vitrified bioactive composition in a stomach of the biological subject, because a pH of the stomach is less than a pH of about 5.

The vitrified bioactive composition may be released from the enterically encapsulated vitrified bioactive composition in a gut of the biological subject when the enterically encapsulated vitrified bioactive composition experiences a pH greater than a pH of about 6. The vitrified bioactive composition that is released in the gut of the biological subject may adhere to the intestinal mucus, rehydrate, resuscitate, and proliferate. The bioactive composition may be released over a duration from the vitrified bioactive composition. The bioactive composition that is released in the gut of the biological subject may release at least one of viable and healthy microbes, cells, tissue, vaccines, probiotics, antibiotics, peptides, RNAi, or mRNA. Prebiotics may be included in the bioactive composition to provide energy to nourish the bioactives.

In some embodiments, the biological subject may be a plant, a seed, or a soil. The applying may include spraying a solution containing particles and/or a powder of enterically encapsulated vitrified bioactive composition on the plant, seed, or soil.

In accordance with another embodiment of the invention, a composition for application to a microbiome of a biological subject includes a bioactive composition. The bioactive composition may include bioactives of at least one of microbes, cells, tissue, vaccines, probiotics, antibiotics, vitamins, peptides, RNAi, or mRNA. The bioactive composition may include a vitrifying mixture. The vitrifying mixture may include intrinsically disordered proteins and saccharides. The bioactive composition is preserved in the vitrifying mixture to form a vitrified bioactive composition.

The composition may include an enteric encapsulant. The enteric encapsulant may be configured to encapsulate the vitrified bioactive composition. The enteric encapsulant may be configured to protect the vitrified bioactive composition from a first set of environmental conditions. The enteric encapsulant may be configured to release the vitrified bioactive composition in a second set of environmental conditions. The bioactive may be freeze dried in preparation for being preserved in the vitrifying mixture. The bioactive composition further comprises a probiotic and/or an antibiotic.

The vitrified bioactive composition may be incorporated in a skin care lotion. The vitrified bioactive composition may be incorporated in a wound healing medication.

The intrinsically disordered proteins may be a silk fibroin. The saccharides is may be a disaccharide. In some embodiments, the intrinsically disordered proteins may be a naturally occurring protein. In some embodiments, the intrinsically disordered proteins may be a synthetic polymer.

In accordance with another embodiment of the invention, a method of delivery of a bioactive composition to a biological subject includes provision of a bioactive composition. The bioactive composition includes bioactives. The method includes preservation of the bioactive composition. Preserving the bioactive composition includes vitrification of the bioactive composition in a vitrifying mixture of intrinsically disordered proteins and saccharides to produce a vitrified bioactive composition. The vitrified bioactive composition is configured to release the bioactive composition from the vitrifying mixture. The method includes provision of an enteric encapsulation. Providing an enteric encapsulation includes encapsulating the vitrified bioactive composition in the enteric encapsulation. The method includes administration of the enterically encapsulated vitrified bioactive composition to the biological subject. Administration includes applying the enterically encapsulated vitrified bioactive composition to a microbiome of the biological subject.

In some embodiments, the enteric encapsulation includes encapsulating the vitrified bioactive composition in a capsule comprising an enteric polymer. In some embodiments, the enteric encapsulation includes coating particles of vitrified bioactive composition with an enteric material. In some embodiments, the enteric encapsulation includes coating tablets of vitrified bioactive composition with an enteric material.

The bioactives comprise at least one of microbes, cells, tissue, vaccines, probiotics, antibiotics, vitamins, peptides, RNAi, or mRNA. The intrinsically disordered proteins may be a tardigrade intrinsically disordered protein, silk fibroin, or regions of tropoelastin, and the saccharides may be a disaccharide.

In accordance with another embodiment of the invention, a method of method of preserving a bioactive composition includes the formation of a vitrifying solution. Formation of the vitrifying solution includes the provision of a liquid, and the addition of intrinsically disordered protein and a saccharide to the liquid. The intrinsically disordered protein and the saccharide are mixed in the liquid to form a the vitrifying solution. A bioactive composition is added to the vitrifying solution, and the bioactive composition is mixed in the vitrifying solution. The liquid is removed from the bioactive composition in the vitrifying solution. The liquid may include water or an alcohol.

In some embodiments, the method further includes the heating of the bioactive composition in the vitrifying solution to a temperature of between 20 degrees Celsius and 125 degrees Celsius. In some embodiments, the method further includes the cooling of the bioactive composition in the vitrifying solution to a temperature of between 0 degrees Celsius and −80 degrees Celsius.

In some embodiments, removing the liquid comprises at least one of air-drying, freeze-drying, vacuum drying, spray drying, or heat-drying.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1A schematically shows an embodiment of a process to prepare a vitrified bioactive composition according to illustrative embodiments;

FIG. 1B schematically shows an embodiment of a structure of a vitrified bioactive composition according to illustrative embodiments;

FIG. 1C schematically shows an embodiment of a process to prepare an enterically encapsulated vitrified bioactive composition according to illustrative embodiments;

FIG. 2 schematically shows some potential embodiments of applications for vitrified bioactive compositions according to illustrative embodiments;

FIG. 3 shows an embodiment of a method of delivery of a bioactive composition in accordance with illustrative embodiments;

FIG. 4 shows an embodiment of a method of delivery of a bioactive composition in accordance with illustrative embodiments;

FIG. 5A schematically shows an embodiment of a process to preserve a bioactive composition according to illustrative embodiments; and

FIG. 5B schematically shows an embodiment of a process to prepare a vitrifying solution according to illustrative embodiments.

### DESCRIPTION OF ILLUSTRATIVE EMBODIMENTS

Certain exemplary embodiments will now be described to provide an overall understanding of the principles of the structure, function, manufacture, and use of the systems, devices, and methods disclosed herein. One or more examples of these embodiments are illustrated in the accompanying drawings. Those skilled in the art will understand that the systems, compositions, and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure.

Live microbial cultures (e.g., natural or engineered) may be used to improve human, animal, and plant health by producing beneficial proteins and enzymes. A formulation of a protein with intrinsically disordered regions and polysaccharides/disaccharides/monosaccharides or a protein with intrinsically disordered regions (synthetically produced or naturally found) alone can be used to nourish, stabilize live cultures (bacteria, protists, archaea, fungi for humans and animals), improve performance of prebiotics and probiotic fractions, and control release of payloads. This material can be used to keep the microbes alive in liquid formulation, in freeze dried state and in dry powder or films.

Similarly, to how tardigrades survive desiccation through extracellular vitrification using intrinsically disordered proteins and disaccharides can preserve and protect microbes for an extended period of time. The formulation may also provide added benefits to the skin and gut during the delivery process. Inventor initial test materials are expected to include a solution of silk fibroin extracted from Bombyx Mori (which is a protein that has a disordered region) and trehalose (a disaccharide). However, other disordered proteins and disaccharides/monosaccharides (synthetic or biologically acquired) may be used. Accordingly, discussion of those specific materials is for exemplary purposes only and not intended to limit various embodiments of the invention.

Engineered microbes, probiotics, and metabolites can be kept stable during manufacturing, storage, and administration by encapsulation in the formulation. This formulation preferably will be applied to the human microbiome, including skin, vaginal, armpit or as an oral ingestible pill, tablet, or solution and may help nourish and protect skin and/or products ingested by humans and animals. Further, prebiotics may be added to increase the efficacy of the microbes (bacteria, protists, archaea and fungi), the prebiotics and materials would provide the energy to nourish the microbes.

In illustrative embodiments, methods to prepare, preserve, and deliver is bioactive components (e.g., bioactives) for treating animals and plants is disclosed. Bioactives comprise at least one bioactive component (e.g., a bioactive). The bioactives may be prepared, preserved, and delivered without killing (e.g., deactivating) the living portions of the bioactives so that the bioactives may be delivered to the recipient with full potency. The bioactive and a bioactive composition may be preserved by vitrifying the bioactive and/or bioactive composition in a vitrifying mixture of intrinsically disordered proteins and saccharides to produce a vitrified bioactive composition. In some embodiments, the vitrified bioactive composition comprises the formulation described above. The vitrified bioactive composition may be encapsulated within an enteric polymer encapsulation that is configured to protect the vitrified bioactive composition in some environments, and release the vitrified bioactive composition in other environments. In embodiments, the vitrified bioactive composition encapsulated within an enteric polymer encapsulation comprises the formulation described above.

Using the combination of vitrifying the bioactive composition, and encapsulating the vitrified bioactive composition in an enteric polymer, bioactives and/or bioactive compositions may be preserved alive, and then delivered to a biological subject under a predetermined set of environmental conditions. In some embodiments, the encapsulated vitrified bioactive composition (e.g., the formulation) may be delivered to a human being or an animal by an orally ingestible pill, capsule, and/or solution. In some embodiments, the encapsulated vitrified bioactive composition may be delivered to a plant, seed, and/or soil by applying powder, and/or pellets, of the encapsulated vitrified bioactive composition by spraying a solution containing particles and/or powders of enterically encapsulated vitrified bioactive composition on the plant, seed, or soil.

Bioactives

In some embodiments, the bioactives (e.g., bioactive substances, or bioactive components) may include microbes, cells, tissue, vaccines, probiotics, antibiotics, vitamins, and mRNA. The bioactive substances may interact with a biological subject, such as a human being, an animal, or a plant.

In some embodiments, the bioactives may be prepared as a bioactive composition which may include more than one bioactive substance. The bioactive components may also include metabolites and/or preservatives. The addition of the metabolites and/or preservatives may enhance the effectiveness of the of the bioactive composition.

In some embodiments, the bioactives and/or bioactive compositions may include fungus, a eubacterium, a eukaryotic organism, or an archaebacteria.

In some embodiments, the bioactives and/or bioactive compositions may be anaerobic, aerobic, spore-forming, vegetative, native to a human microbiome, native to an animal microbiome, non-native to the human microbiome, non-native to the animal microbiome, native to a plant microbiome, non-native to the plant microbiome, an engineered version of the bioactive composition that is non-native to the human microbiome, or an engineered version of the bioactive composition that is non-native to the animal microbiome.

In some embodiments, the bioactives and/or bioactive compositions may include one or more of a bacteria, a metal, an enzyme, or a biologic. For example, the metal may include one or more of an alkali metal, an alkaline earth metal, or a transition metal. In some embodiments, the biologic may be an insulin glargine, infliximab, rituximab, etanercept, adalimumab, monoclonal antibodies, trastuzumab, or other biologics.

In some embodiments, the bioactive may be an oligonucleotide, such as an RNA. The RNA may be tRNA, mRNA, rRNA, snRNA, srpRNA, gRNA, TERC, SL RNA, crRNA, miRNA, siRNA, or eRNA.

In some embodiments, the bioactive may be an enzyme (i.e., an RNase or a DNase), a fatty acid, a sugar (e.g., an alcohol sugar), or a mineral. For example, the enzyme may include erepsin maltase, lactase, sucrase, disaccharidases, lingual lipase, lysozymes, salivary amylase, pepsin, gastric lipase, other lipases, hydrochloric acids, intrinsic factors, mucins, gastrins, trypsinogen, ductal cells, carboxypeptidase, elastases, and the like.

In some embodiments, an additive may be added to the bioactive composition. The additive may be at least one of a coloring agent, a chelator, a ligand, an antimicrobial, a filler, a scent, or a flavor. For example, the coloring agent may be allura red, Ponceau 3R, amaranth, erythrosine, indigotin, Light Green SF, Naphtha! yellow, Orange 1, quinoline yellow, tartrazine, an E1 suit (e.g., E100, E161b, etc.), an anthocyanin, a betacyanin, a carotenoid, or a phenolic. In some embodiments, the chelator may be ethylenediaminetetraacetic acid (EDTA), transferrin, or desferrixoxamine.

In some embodiments, the microbial may be acetic acid, benzoic acid, natamycin, nisin, nitrate, nitrite, propionic acid, sorbic acid, sulfite, or sulfur dioxide. In other examples, the filler may be cellulose.

In some embodiments, the bioactive may be at least one of a vitamin, a nutrient, an antioxidant, and a protein. In some examples, the protein may be a peptide, an amino acid, (e.g., a post-translated amino acid), or a synthetic amino acid. A nutrient may be defined as a mineral, protein, carbohydrate, fat, Q10, glutathione, lithium, probiotic, glycine, DHA, flavonoid, or others. An antioxidant may include vitamins C and E, selenium, carotenoids, thiols, catalase, superoxide dismutase, uric acid, and ubiquinol.

In some further embodiments, the bioactive may be at least one of a green tea extract, a rosemary extract, a phenolic antioxidant, catechin, acerola, tocopherol, chamomile extract, malphigia emarginata, Camellia sinensis, epicatechin, epigallocatechin, gallochatechin, epigallocatechin gallates, vitamin A, vitamin E, and/or vitamin C.

In some embodiments, the additive may be mixed with an accelerant or an excipient. For example, the additive may be mixed with polyethylene glycol or xylitol. In some further aspects, the additive may be emulsified with the accelerant or excipient and mixed into a silk fibroin solution.

Vitrification

In embodiments, the bioactives and/or bioactive compositions are mixed with a vitrifying combination of a structural protein and a saccharide. The structural protein and the saccharide may be mixed with a solution to provide a vitrifying solution. Vitrification is the transformation of substance into a non-crystalline amorphous (e.g., glass) substance. Together with the bioactive and/or a bioactive composition, the intrinsically disordered proteins and saccharides undergoes vitrification under predetermined conditions.

For example, a slow annealing process at pressures below atmospheric pressure may include starting with the combination of the bioactive and/or bioactive composition and the intrinsically disordered proteins and saccharides to form the vitrifying solution at room temperature and pressure in a reaction vessel. The reaction vessel may be slowly evacuated at room temperature over 2-12 hours. The slow evacuation process in a reaction vessel may lead an is amorphous substance that has a highly ordered local layering of silk protein and disaccharides on the surface of the bioactive and/or bioactive composition (which herein is referred to as a “vitrified bioactive composition”). In some other aspects of the embodiment, the silk fibroin solution may be dried via air-drying, freeze-drying, vacuum drying, spray drying, or heat-drying.

This highly ordered, layered substance is preferably dense enough to significantly slow the rate of exchange of oxygen from the ambient air to the vitrified bioactive composition, and vice versa. This vitrification process effectively pauses the bioactive and/or bioactive composition (e.g., lowering its metabolism and putting it in stasis) without killing it. Additionally, vitrification protects anaerobic bioactives and/or anaerobic bioactive compositions (e.g., substances that cannot survive in the presence of oxygen) from dying under normal temperature and pressure conditions due to exposure to oxygen.

When vitrifying anaerobic bioactive, and/or bioactive compositions the entire annealing process must take place in an anaerobic reaction vessel, under oxygen-free conditions. The anaerobic microbe should only be exposed to oxygen (e.g., air) after the mixture is completely annealed into the vitrified bioactive composition. The materials used in the vitrification process are generally regarded as safe therefore can be ingested by humans.

Intrinsically Disordered Proteins

As described above, the vitrifying mixture includes intrinsically disordered proteins and saccharides. The intrinsically disordered proteins are structural proteins and may be a naturally occurring protein; or a synthetic polymer. An intrinsically disordered protein (IDP) is a protein that lacks a fixed or ordered three-dimensional structure, typically in the absence of its macromolecular interaction partners.

In some embodiments, the structural protein (e.g., intrinsically disordered protein) is a protein composed of repetitive units that form ordered structures (e.g., beta-sheet) staggered by a non-repetitive region of the amino acid chain that form non-ordered secondary and tertiary structures. In some embodiments, the structural protein is selected from silk fibroin, suckerin, or keratin.

In some embodiments, the structural protein is silk fibroin. In some embodiments, the silk fibroin is extracted from Bombyx mori cocoons. Silk fibroin is a structural protein that is well known for its application in textiles, and that has been reinvented as a naturally-derived technical material with applications in regenerative medicine, drug delivery, implantable optoelectronics, and food coating.

Silk fibroin is a structural protein that may be produced and extracted from silkworm, spiders, or other insects. It can also be otherwise generated synthetically. Silk fibroin is naturally produced by species such as, without limitation, Antheraea mylitta; Araneus bicentenaries; Araneus ventricosus; Bombyx mori; Bombyx mandarins; Galleria mellonella; Nephila clavipes; Nephila madagascariensis; and Tetragnatha versicolor. Silk fibroin's unique properties are derived from its structure, consisting of hydrophobic blocks separated by hydrophilic spacers. In its natural state, silk fibroin is organized in beta-sheets, which are formed by highly ordered crystalline regions alternated by amorphous regions. This unique structure results in high levels of strength and toughness for silk fibroin-based materials. The wide range of forms into which silk fibroin solution can be processed make it attractive for several high-tech applications, including scaffolds for tissue engineering, bone screws for fixation, and drug depots for therapeutic delivery.

Among other insects, the Bombyx mori silkworm starts its life in a cocoon. A single cocoon can be made up of hundreds of meters of silk. The cocoon itself is made up largely by two proteins: fibroin and sericin. With the other component parts, the fibroin helps regulate gas diffusion in and out of the cocoon. Similarly, such regulation is achievable with silk fibroin-based materials. This is applicable to preserving bioactives given that the main modes of spoilage relate to oxidation, degradation, and dehydration. When applied to bioactive components, the tunability of silk fibroin allows for its versatility of application across different bioactive types. Silk fibroin forms polymeric coatings with varying degree of solubility and breathability. Fine-tuning is enabled by different production parameters such as concentration, shear force, additives, temperature, and postprocessing. By tuning these parameters, silk-fibroin structural organization may be controlled. Structural differences ultimately affect crystallinity, elasticity, solubility and porosity which in him alter the gas and water diffusion behavior, as well as reduce microbial growth rates. Silk coatings can be designed for different storage conditions or products of interest.

The silk fibroin structural protein is purified from cocoons into a water suspension using a water-based process that uses chaotropic ions as LiBr to break the inter- and intra-molecular hydrogen bonds that crosslink silk fibroin molecules into fibers. Upon removal of the ions via dialysis, silk fibroin has the form of nanomicelles in water suspension that are stable for a period of time that ranges from days to months, depending on concentration, pH and molecular weight.

Material assembly is driven by water removal and formation of new intra- and inter-molecular hydrogen bonds. This process can be engineered to obtain several materials formats, including transparent, robust membranes that have been used to extend the shelf-life of perishable crops. The combination of a di-block copolymer-like structure with hydrophobic repetitive amino acid is sequences spaced out by hydrophilic, negatively charges non-repetitive sequences make silk fibroin polymorphic, because the protein can be obtained in random coil or beta-sheet rich structures, enabling the fabrication of silk materials that are water soluble or water-insoluble, respectively. Silk fibroin structure also provides a distinct environment that can preserve labile compounds ranging from antibiotics to growth factors, enzymes and viruses by mitigating oxidative stress, providing sufficient hydration and maintaining biomolecules configuration in anhydrous conditions.

Silk fibroin extracted from Bombyx Mori (e.g., silk moth) is a naturally occurring protein. Another naturally occurring protein is include silk fibroin extracted from tardigrades. Tardigrades are microscopic aquatic animals that exhibit remarkable resistance to environmental stresses such as extreme temperature, radiation, high pressure, or absence of oxygen or water. In response to such stress, tardigrades transform into a characteristic turn state that can survive for long periods of time. Although the molecular mechanisms that confer this protection against extreme physiological stress remains poorly understood, the importance of two families of tardigrade-unique intrinsically disordered proteins (cytosolic abundant heat-soluble (CAHS) proteins and secreted abundant heat-soluble (SAHS) proteins) have been identified. These tardigrade-unique intrinsically disordered proteins include cytosolic abundant heat-soluble (CAHS) proteins, and secreted abundant heat-soluble (SAHS) proteins. These proteins are collectively known as tardigrade-specific disordered proteins (TDPs). RNA interference experiments have demonstrated that TDPs were required for desiccation tolerance in tardigrades, while heterologous expression of certain TDPs demonstrated their ability to increase desiccation tolerance in both yeast and E. coli.

In some embodiments, elastins may be incorporated as the intrinsic disordered proteins (e.g., IDPs). Elastin is arranged as fibers and/or discontinuous sheets in the extracellular matrix where it confers the properties of stretching and elastic recoil. Elastin is a protein synthesized by fibroblasts in the form of a precursor monomer known as tropoelastin. The monomers are polymerized in the extracellular matrix by the enzyme lysyl oxidase, with extensive cross-linking of lysine amino acid side chains. Deposition of elastin in the form of fibers requires the presence of a template of microfibrils of the structural glycoprotein fibrillin and associated glycoproteins. These become incorporated around and within the ultimate elastic fiber. Tropoelastin is the soluble precursor of elastin that is secreted as a 60-70-kDa monomer by elastogenic cells such as fibroblasts, lung alveolar cells, chondrocytes, and vascular smooth muscle cells. Upon secretion, tropoelastin has the propensity to self-aggregate through a process known as coacervation. The alignment of the tropoelastin aggregates occurs on the cell surface and is mediated by interactions with cell surface proteoglycans.

Elastin is the name of both the fiber and the polymerized protein. There are also two named related fibers, oxytalan and elaunin, which have more fibrillin and less polymerized tropoelastin than generic elastin. In some embodiments, tropoelastin is used an IDP with saccharides to form the vitreous mixture.

In some embodiments, synthetic polymers may be incorporated as the intrinsically disordered protein (e.g., IDP). For example, elastin-like polypeptides (e.g., ELPs) are a family of lab-designed intrinsically disordered proteins which, while not normally found in nature, share many of the same characteristics as natural intrinsically disordered proteins, including temperature responsiveness and salt/pH sensitivity. The ELPs may be genetically encoded as a sequence of amino acids. They may be fused to various ribonucleic acid (e.g., RNA) binding is proteins and form synthetic IDP-RNA-rich granules. These synthetic granules can be engineered to bind with deoxyribonucleic acid (DNA) and RNA. In some embodiments, natural IDP-RNA granules may be replicated using synthetic IDP-RNA granules to enable delivery of DNA and RNA into natural systems as therapeutic treatments for disease states. The ELPs can be fused to a protein targeting specific mRNA to modulate gene expression by sequestering target mRNA using ELPs' temperature-responsive phase separation activity.

Synthetic polymers may be incorporated as the IDP to improve specific properties of vitrified bioactive compositions. For example, certain synthetic IDPs may have increased temperature tolerance. Other synthetic IDPs may have increased radiation tolerance.

Saccharides

As described above, saccharides may be combined with an intrinsically disordered protein to form the vitrifying mixture. In some embodiments, the saccharide is a disaccharide. The disaccharide may be selected from trehalose, sucrose, lactose, or maltose. In some embodiments, the disaccharide is trehalose.

Trehalose is a nonreducing disaccharide in which the two glucose units are linked via an a,a-(1,1)-glycosidic bond. This disaccharide has been isolated from all domains of life including plants, animals, fungi, yeast, archaea, and bacteria. Trehalose is also industrially produced as it is used in the food, cosmetics, and pharmaceutical industries. This disaccharide can play important and different roles in cells, such as, as signaling molecule, carbohydrate reserve, and protectant against various stresses (e.g., drought, cold, and salt.) Accumulation of trehalose occurs both intra- and extra-cellularly.

Forming Vitrified Bioactive Compositions

As described above, in some embodiments, a bioactive composition (e.g., formulation) is combined with a mixture of intrinsically disordered proteins (e.g., IDPs), such as silk fibroin, and saccharides, such as disaccharides, to produce a vitrified bioactive composition. The combination of IDPs and saccharides may be mixed in a solvent to prepare a vitrifying solution. The vitrification process provides a microenvironment for the bioactive materials (e.g., bioactives) bioactive composition. Solvents that may be used include water and alcohols.

The detailed formation mechanism of a saccharide-driven cellular protection with includes the saccharide (e.g., trehalose) forming a glass-like matrix within cells, physically preventing protein denaturation of the IDPs, protein aggregation, and membrane fusion. The water in hydrogen bonds between water and cellular components in a solution of IDPs and saccharides is replaced by trehalose as cells dry, which would also prevent protein denaturation of the IDPs, as well as, aggregation, and membrane fusion of the saccharides. That is, the replacement of hydrogen bonds between silk fibroin molecules and water with inter- and intra-molecular hydrogen bonds may be thermodynamically favorable even in the presence of trehalose, and that the protein can undergo structural reconfiguration, water loss and volume change despite the trehalose-induced vitrification of the protein.

In some embodiments, the vitrified bioactive composition comprises an IDP to saccharide ratio (e.g., IDP:saccharide) from about 10:1 to about 1:10, respectively. In some embodiments, the vitrified bioactive composition comprises an IDP to saccharide ratio from about 5:1 to about 1:5, respectively. In some embodiments, the vitrified bioactive composition comprises an IDP to saccharide ratio from about 3:1 to about 1:3, respectively. In some embodiments, the vitrified bioactive composition comprises an IDP to saccharide ratio is about 1:1. In some embodiments, the vitrified bioactive composition comprises a mixture of multiple IDPs and saccharides.

The preservation of biomolecules in silk fibroin formulations correlates with matrix β-relaxation, as it does in sugar-based dry formulations. The inclusion of sugars like sucrose in silk fibroin-based materials enhances the protein stabilizing performance as they can act as antiplasticizers that suppress β-relaxation and decelerate degradation rates. Trehalose does not interfere with silk fibroin assembly as its addition to silk fibroin suspensions does not impart any modification to the random coil structure assumed by silk fibroin, nor does it drive protein assembly.

In some embodiments, an additive may be added to the vitrifying solution. These additives may include at least one of a sugar, a plasticizer, or a crosslinking agent. For example, the sugar additive may be a sugar-al, a poly-al, or a hygroscopic polymer (e.g., polyethylene glycol). In some embodiments, the sugar additive is a crosslinking agent, the crosslinking agent may be photoreactive.

Preparations of Bioactive Compositions with IDPs and Saccharides

The preparation processes of combining the bioactive components with vitrifying solutions (e.g., a mixture of IDPs and saccharides in a solvent) into vitrified bioactive compositions to preserve and protect the bioactive compositions from spoilage can be accomplished by many approaches.

In some embodiments, the vitrifying solution may be deposited onto the bioactive composition via spray-coating. Alternatively, the vitrifying solution may be deposited onto the bioactive composition via dip-coating.

In some aspects, the vitrifying solution may not be annealed after or before deposition. In some embodiments, the bioactive composition may include is multiple layers of silk fibroin/saccharides deposited by the vitrifying solution. For example; the bioactive could be sprayed with the vitrifying solution, dried, and then sprayed once more. This can happen any number of times to add thickness and additional layers. In some further embodiments, the bioactive composition may comprise of multiple layers, with each layer serving a function. For example, the bioactive may be coated with silk fibroin/saccharide. Then, the silk fibroin/saccharide-coated bioactive may be itself coated by another coating that is hydrophobic or water-tight such that water may not permeate the outer layer and reach the inner silk fibroin/saccharide layer.

In some further embodiments, a tablet-coating may be utilized, where the silk fibroin/saccharide layer is coated while in an industrially-relevant drum. Tablet coating may additionally be utilized, as well as film-coating.

In some embodiments, the preservation of the bioactive composition is directed to a bioactive composition comprising silk fibroin and saccharides, wherein the silk fibroin was previously in a powder form or in a solution in which powdered silk fibroin has been reconstituted within a solvent.

In some embodiments, the preservation of the bioactive composition is directed to a solution containing silk fibroin with saccharides. In some embodiments, no more than 25% of the solution may comprise silk fibroin fragments. In some further embodiments, no more than 10% of the silk fibroin fragments may have a molecular weight of over 400 kilodaltons (kDa). In some alternative further aspects, no more than 45% of the silk fibroin fragments may have a molecular weight of over 300 kDa.

In some embodiments, the preservation of the bioactive composition is directed to a bioactive composition comprising a substrate, such as a powder, pill, tablet, capsule, and the like. The substrate may be coated with at least one silk fibroin/saccharide layer. In some embodiments, the thickness of the at least one silk fibroin/saccharide layer may range from about 12 nm to about 180 μm (e.g., microns).

In some embodiments, the preservation is directed to a method of preparing a bioactive composition. In some embodiments, the method may comprise spray-coating the bioactive composition with a solution containing silk fibroin fragments. In some embodiments, the silk fibroin fragments may range from 10 kDa to 600 kDa.

In some embodiments, the preservation of the bioactive composition is directed to a method of preparing a bioactive composition. In some embodiments, the bioactive composition may comprise dip-coating the bioactive composition with a solution comprising silk fibroin fragments. In some embodiments, the silk fibroin fragments may range from 10 kDa to 600 kDa.

In some embodiments, the preservation is directed to a method of preparing a bioactive composition. In some embodiments, the bioactive composition may comprise mixing the bioactive composition with silk fibroin fragments. In some embodiments, the silk fibroin fragments may range from 10 kDa to 600 kDa.

In some embodiments, the preservation of the bioactive composition is directed to a method of preparing a bioactive composition. In some embodiments, the bioactive composition is only mixed with, or covered by a silk fibroin. That is, in some embodiments, the vitrified bioactive composition does not include saccharides.

In some embodiments, the preservation of the bioactive composition is directed to a method of preparing a bioactive composition. In some embodiments, the bioactive composition is only mixed with, or covered by a saccharides. That is, in some embodiments, the vitrified bioactive composition is does not include silk fibroins.

Enteric Polymers

In a general sense, an enteric coating is a polymer barrier applied to a bioactive component that prevents its dissolution or disintegration in a given environment, but allows dissolution or disintegration in a different environment. A common example is the application of a polymer barrier to an oral medication that prevents its dissolution or disintegration in a gastric environment. That is, the enteric coating may keep the polymer-coated oral medication from dissolving or disintegrating in the stomach, so that the medication may be delivered to a portion of the digestive track of a subject that is after the stomach. Some drugs are unstable at the pH of gastric acid (pH about 1-2), and need to be protected from degradation. Enteric coatings are also an effective methods to obtain drug targeting (such as gastro-resistant drugs). Other drugs, such as some anthelmintics, may need to reach a high concentration in a specific part of the intestine. Enteric coatings may also be used during studies as a research tool to determine drug absorption. Enteric-coated medications pertain to the “delayed action” dosage form category.

Tablets, mini-tablets, pellets, and granules (usually filled into capsule shells) are the most common enteric-coated dosage forms. The enteric polymer can be applied to a surface of a tablet, mini-tablet, pellet, and a powder. Each tablet, mini-tablet, pellet, and powder is coated with the enteric polymer coating in a configuration that allows the each particle in the powder, pellet, mini-tablet, and tablet to be fully covered. That is, regardless of the size of the specific bioactive component, a specific coating may be a polymeric enteric coating.

The enteric polymer may also be a coating on a capsule. In this embodiment, the vitrified bioactive composition is encapsulated in a capsule with an enteric polymer coating. In embodiments, the vitrified bioactive composition is encapsulated in a capsule made entirely with an enteric polymer.

By preventing the bioactive composition from dissolving into the stomach, an enteric coating may protect gastric mucosa from the irritating effects of the medication itself. When the drug reaches the neutral or alkaline environment of the intestine, its active ingredients can then dissolve and become available for absorption into the bloodstream. Bioactive compositions that have an irritant effect on the stomach, can be coated with a substance that will dissolve only in the small intestine. Likewise, certain groups bioactive composition may be acid-activated. For such bioactive composition, enteric coating added to the formulation tends to avoid activation in the mouth and esophagus.

Materials used for enteric coatings include fatty acids, waxes, shellac, plastics, and plant fibers. Conventional materials used are solutions of film resins.

Microbiome

The microbiome is defined as a characteristic microbial community occupying a reasonable well-defined habitat which has distinct physio-chemical properties. The microbiome not only refers to the microorganisms involved but also encompass their theatre of activity, which results in the formation of specific ecological niches. The microbiome, which forms a dynamic and interactive micro-ecosystem prone to change in time and scale, is integrated in macro-ecosystems including eukaryotic hosts, and here crucial for their functioning and health.

The microbiota, which is different from the microbiome, but is interconnected, consists of the assembly of microorganisms belonging to different kingdoms (prokaryotes (bacteria, archaea), eukaryotes (algae, protozoa, fungi, etc.), while “their theatre of activity” includes microbial structures, metabolites, mobile genetic elements (such as transposons, phages, and viruses), and relic DNA embedded in the environmental conditions of the habitat.

In some embodiments, a bioactive and/or bioactive composition is delivered to the microbiome. The bioactive/bioactive composition may contain microbiota that is specific to the microbiome to which it is being delivered.

Forms of Delivery

Using the combination of vitrifying the bioactive composition, and encapsulating the vitrified bioactive composition in an enteric polymer, bioactives and/or bioactive compositions may be preserved alive, and then delivered to the microbiome of biological subject under a predetermined set of environmental conditions.

In some embodiments, methods are described to protect, preserve and control the release of microbes and other bioactive components to various microbiomes on the skin, mouth, vagina, and gut of humans and animals. Some embodiments may serve other parts of the body. Among other states, some embodiments may be in the form of a formulation (e.g. vitrified bioactive composition) in a liquid state, a freeze dried, a gel state, or a film state and the formulation may be constituted of intrinsically disordered proteins and disaccharides and prebiotics that engineer the environment of the microbes and other bioactive components to ensure it has nutrients and is protected from desiccation, temperature, oxygen and water vapor stress. In some embodiments, methods are described to protect, preserve and control the release of microbes and other bioactive components to various microbiomes on plants, seeds, and is soils.

In some embodiments, the encapsulated vitrified bioactive composition (e.g., the formulation) may be delivered to a human being or an animal by an orally ingestible pill, capsule, powder, pellet, and/or solution. The bioactive composition that is released in the gut of the biological subject releases at least one of viable and healthy microbes, cells, tissue, vaccines, probiotics, antibiotics, peptides, RNAi, or mRNA.

In some embodiments, the encapsulated vitrified bioactive composition may be delivered to a plant, plant seed, and/or soil by applying a solution, powder, and/or pellets, of the encapsulated vitrified bioactive composition. The encapsulated vitrified bioactive composition may be delivered to a plant, plant seed, and/or soil by spraying a solution containing particles and/or powders of enterically encapsulated vitrified bioactive composition on the plant, seed, or soil.

In some embodiments, the vitrified bioactive composition may be delivered to a human being, animal, and/or plant without encapsulation by an enteric polymer.

In some embodiments, the vitrified bioactive composition may be delivered to a human being, animal, and/or plant with encapsulation by an enteric polymer.

Among other states, some embodiments of the bioactive composition may be in the form of a formulation in a liquid state, a freeze dried, a gel state, or a film state and the formulation may be constituted of intrinsically disordered proteins and disaccharides and/or prebiotics that engineer the environment of the microbe (e.g., bioactive) to ensure it has nutrients and is protected from desiccation, temperature, oxygen, and water vapor stress.

In embodiments, the vitrified bioactive compositions may include plant microbes, and other plant bioactives, as a biological fertilizer as a substitute for the previous plant growth benefits of herbicides, pesticides, and synthetic fertilizers. The use of silk/saccharide dry films as seed coatings can be used to localize and quantify delivery of plant microbes that can mitigate plant stress, and soil salinity. A number of different types of microbes and other plant bioactives can be delivered using the same technology.

In some embodiments, the bioactive composition may be implemented as a biodegradable material having an intrinsically disordered protein (IDP) and a disaccharide, which will be used to preserve commensal gut microbes at or near room temperature under aerobic conditions. An IDP is any protein that largely lacks secondary structure in an aqueous solution, such as silk fibroin from Bombyx mori. The encapsulated microbes can be fungal, eubacterial, protist, or archaeabacterial in nature. They can be anaerobic or aerobic (intolerant or tolerant of oxygen, respectively), spore-forming or vegetative, native to the human or animal microbiomes, non-native but proven to be beneficial to the human or animal microbiome, or an engineered version of the previous two categories of microbes. The mixture may be enhanced with the addition of some metabolites or preservatives.

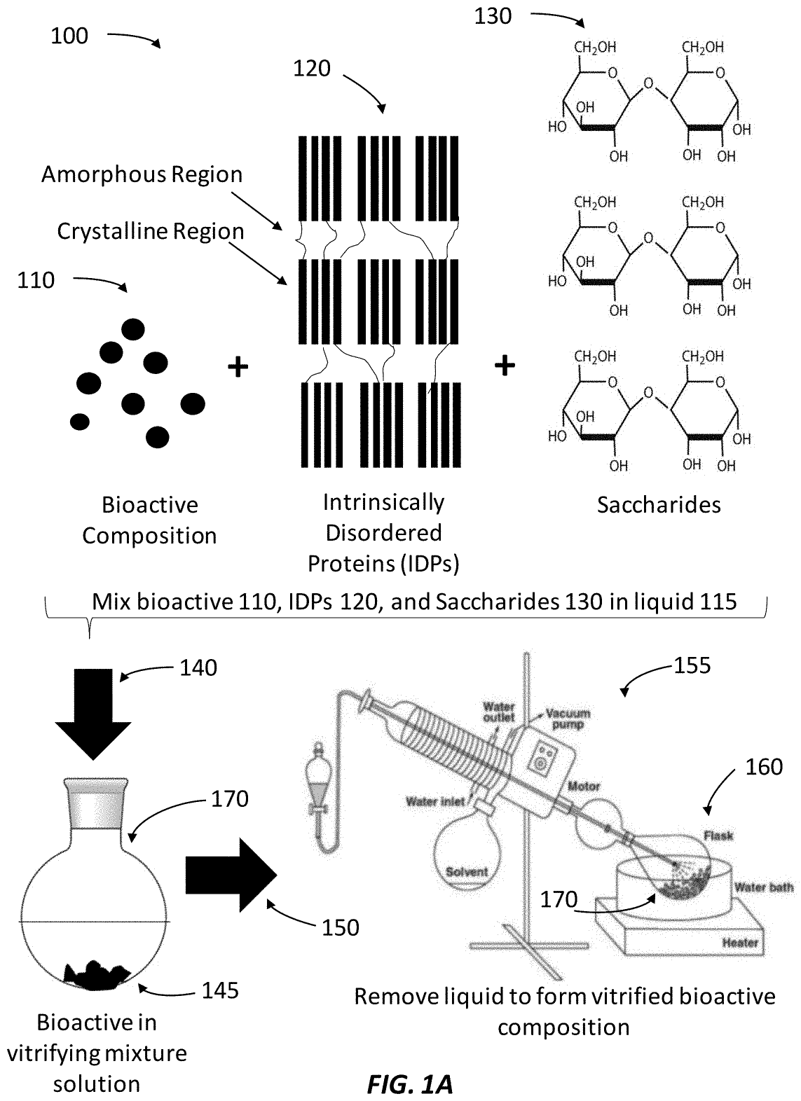

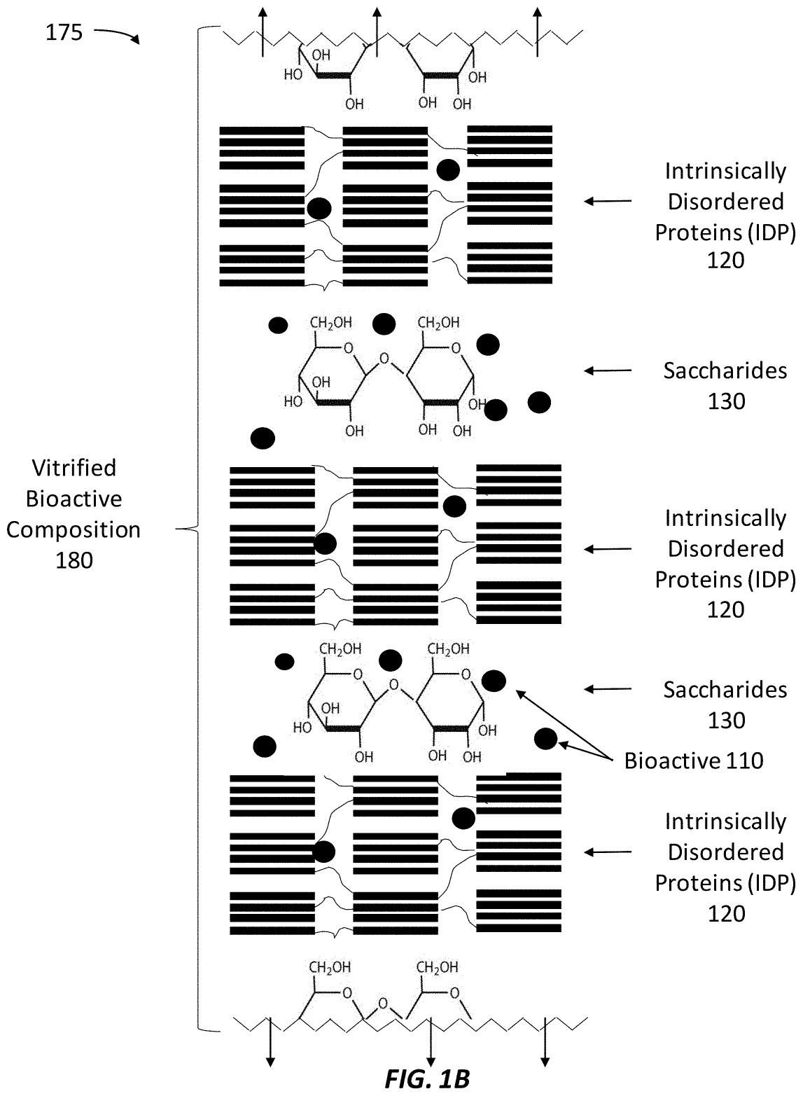

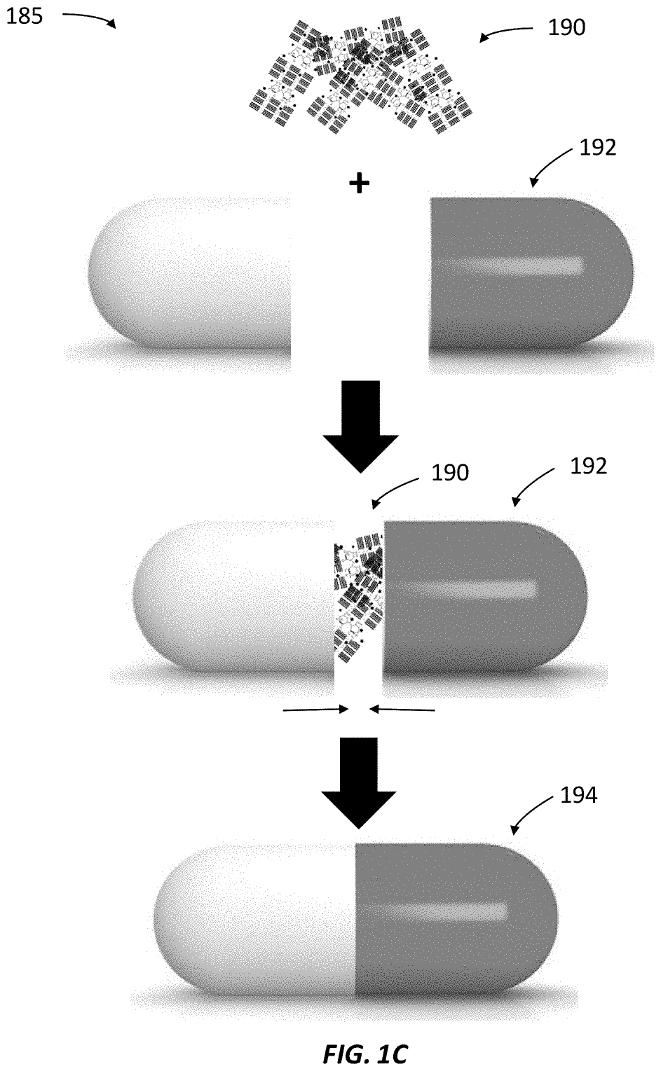

FIG. 1A schematically shows an embodiment of a process to prepare a vitrified bioactive composition comprising one or more bioactive components according to illustrative embodiments. The process 100 preserves a bioactive composition by keeping one or more bioactive components alive and preventing the bioactive(s) from spoilage related to oxidation, degradation, and/or dehydration.

Briefly, a bioactive composition 110, intrinsically disorder proteins (IDPs) 120, and saccharides 130 are added to, and mixed in a liquid 115 to form a bioactive composition in a vitrifying mixture solution 145. The liquid 115 is then removed from to form the vitrified bioactive composition that remains in the container 170.

As shown in FIG. 1A, a bioactive composition 110 is added to a liquid 115. The bioactive composition 110 may include at least one of microbes, cells, tissue, vaccines, probiotics, antibiotics, vitamins, or mRNA.

Further, intrinsically disordered proteins (IDPs) 120 are also included with the bioactive composition 110 in the liquid 115. The IDPs are structural proteins that lack a fixed or ordered three-dimensional structure, typically in the absence of its macromolecular interaction partners. Silk fibroin is a non-limiting example of a structural protein that may be produced and extracted from silkworm, spiders, or other insects. It can also be otherwise generated synthetically.

Silk fibroin is a structural protein that is well known for its application in textiles, and that has been reinvented as a naturally-derived technical material with applications in regenerative medicine, drug delivery, implantable optoelectronics, and food coating. Silk fibroin's unique properties are derived from its structure, consisting of hydrophobic blocks separated by hydrophilic spacers. In its natural state, silk fibroin is organized in beta-sheets, which are formed by highly ordered crystalline regions alternated by amorphous regions. Amorphous regions and crystalline regions are identified in the schematic representation of IDPs in FIG. 1A.

Also, as shown in FIG. 1A, saccharides 130 are added to the intrinsically disordered proteins (IDPs) 120 and the bioactive composition 110 in the liquid 115. The saccharides 130 are for illustrative purposes and are non-limiting, as saccharides may generally be used in making the vitrifying mixture. For example, saccharides may be in the form of polysaccharides, disaccharides, is and/or monosaccharides.

As discussed, the FIG. 1A shows the bioactive composition 110, the IDPs 120, and saccharides 130 being added to 140, and mixed in a liquid 115 to form a bioactive composition in a vitrifying mixture solution 145 in a container 170.

The liquid in the vitrifying mixture solution 145 is removed 150 in a system 155, as schematically shown in a non-limiting illustration, in FIG. 1A. A vacuum may be applied to the container 170 holding the bioactive composition/vitrifying mixture solution 145 to expedite removal of the liquid 115.

In some embodiments, the container 170 with the bioactive composition/vitrifying mixture solution 145 may be heated to facilitate mixing of the components and the formation of the vitrified bioactive composition. The mixture may be heated to between about 20 and 125 degrees C.

In some embodiments, the container with the bioactive composition in a vitrifying mixture solution 145 may be cooled. In particular, the mixture with the bioactive composition/vitrifying mixture solution 145 may be cooled to between 0 and −80 degrees C. In embodiments, the mixture may be cooled until it freezes. In the case of a frozen mixture, a vacuum may be applied to the frozen bioactive composition in a vitrifying mixture solution 145 to remove the liquid that was frozen with the bioactive composition in a vitrifying mixture solution 145. After removal of the liquid that was frozen with the bioactive composition in a vitrifying mixture solution 145, the remaining powder will by the vitrified bioactive composition.

FIG. 1B schematically shows an embodiment 175 of a structure of a vitrified bioactive composition 180 according to illustrative embodiments. The vitrified bioactive composition 180 forms as a complex involving the IDPs 120, the saccharides 130, and the bioactive composition 110. The detailed formation mechanism of the saccharide-driven cellular protection with includes the saccharide forming a glass-like matrix within cells, physically preventing protein denaturation of the IDPs 120, protein aggregation, and membrane fusion. The water in hydrogen bonds between water and cellular components in a solution of IDPs 120 and saccharides 130 is replaced by trehalose as cells dry, which would also prevent protein denaturation of the IDPs 120, as well as, aggregation, and membrane fusion of the saccharides 130. That is, the replacement of hydrogen bonds between the IDPs (e.g., for example, silk fibroin) molecules and water with inter- and intra-molecular hydrogen bonds may be thermodynamically favorable even in the presence of trehalose, and that the protein can undergo structural reconfiguration, water loss and volume change despite the trehalose-induced vitrification of the protein. This vitrified bioactive composition may also be referred to an IDP/saccharide/bioactive complex. Alcohols may also be used a solvent. In particular, ethanol may be used as a liquid in the formation of the vitrifying mixture solution.

It is the formation of the glass-like matrix within the vitrified IDP/saccharide/bioactive complex that preserves the bioactive composition. It is unexpected that particles and/or powders of bioactive components could be preserved by being mixed with, and mixed into an IDP/saccharide glass that protects the bioactive composition from spoilage. In particular, the process of forming the vitrified bioactive composition, as well as the vitrified bioactive composition itself are each preserving and protecting of the bioactive composition.

Once formed, the glass-like IDP/saccharide/bioactive matrix (e.g., vitrified bioactive composition) may be processed further to prepare the vitrified bioactive composition in the form of a powder, pellets, tablets, beads, or the like. The as-processed vitrified bioactive composition may be ground into a powder. In some embodiments, the powder may have a range of particle sizes from about 500 nanometers (e.g., nm) to about 1 millimeter (e.g., mm). In some embodiments, the powder may have a range of particle sizes from about 1 micron to about 500 microns. In some embodiments, the powder may have a range of particle sizes from about 5 microns to about 50 microns.

Powders of the vitrified bioactive composition, whether used as-processed, or as a result of grinding, may be processed into pellets. In some embodiments, the pellets may have a size between about 0.2 mm to about 2.0 mm. The pellets may be formed into tablets and capsules. The pellets may also be incorporated into polymer capsules.

The incorporation of bioactive components in a glass-like matrix is essentially dispersing the powder/particles in a glass-like matrix that protects the powder/particles in a form that can be applied to an exterior of a human being, an animal, and a plant, and can be delivered orally to a human being or an animal. Furthermore, the prepared vitrified bioactive composition can be measured out to be packaged for administration in doses of calibrated amounts.

The formation of the glass-like IDP/saccharide/bioactive matrix (e.g., vitrified bioactive composition) that incorporates a powder/particles of one or more bioactive components into a glass-like matrix differs from simply coating a larger item, such as a vegetable. While coating a vegetable, such as an avocado, with IDP's, saccharides, or a combination of IDPs and saccharides may reduce spoilage of the vegetable, coating the vegetable is different than preserving one or more powdered bioactive components in a glass-like matrix. For example, spray coating or dip coating a vegetable or piece of fruit is not the same as dispersing a powder in a vitrifying solution, and then preparing a powder of the vitrified bioactive composition, for use as a powder, or put into a cream, that can be applied to an exterior of a human being, an animal, and a plant, and can be delivered orally to a human being or an animal. Furthermore, the prepared vitrified bioactive composition can be measured out to be packaged for administration in doses of calibrated amounts. FIG. 1C schematically shows an embodiment of a process to prepare an enterically encapsulated vitrified bioactive composition according to illustrative embodiments. An amount of vitrified bioactive composition 190 is provided for encapsulation. The vitrified bioactive composition 190 may be in the form of a powder, pellets, or beads.

The vitrified bioactive composition 190 may be packaged in a capsule 192 that may be made of, or may be coated by an enteric material. In some embodiments, the enteric material is a polymer. The enteric polymer may also be a coating on the capsule 192. In this embodiment, the vitrified bioactive composition is encapsulated in a capsule with an enteric polymer coating. In embodiments, the vitrified bioactive composition is encapsulated in a capsule made entirely with an enteric polymer.

The enterically encapsulated vitrified bioactive composition 194 may be administered as an oral medication. The enteric encapsulation prevents the dissolution or disintegration of the vitrified bioactive composition in a gastric environment. That is, the enteric coating may keep the polymer-coated oral medication from dissolving or disintegrating in the stomach, so that the medication may be delivered to a portion of the digestive track of a subject that is after the stomach in the digestive system. Some bioactive components are unstable at the pH of gastric acid (pH about 1-2), and need to be protected from degradation. Enteric coatings are also an effective methods to obtain bioactive targeting of bioactive compositions that are targeted at the gut microbiome.

The preparation of enterically encapsulated vitrified bioactive compositions is not limited to encapsulation in capsules. In some embodiments, is the vitrified bioactive compositions may be prepared as tables, pellets, or beads that may be coated with an enteric polymer. Tablets, mini-tablets, pellets, and granules are the most common enteric-coated dosage forms. The enteric polymer can be applied to a surface of a tablet, mini-tablet, pellet, and a powder. Each tablet, mini-tablet, pellet, and powder is coated with the enteric polymer coating in a configuration that allows for each particle in the powder, pellet, mini-tablet, and tablet to be fully covered. That is, regardless of the size of the specific bioactive component, a specific coating may be a polymeric enteric coating.

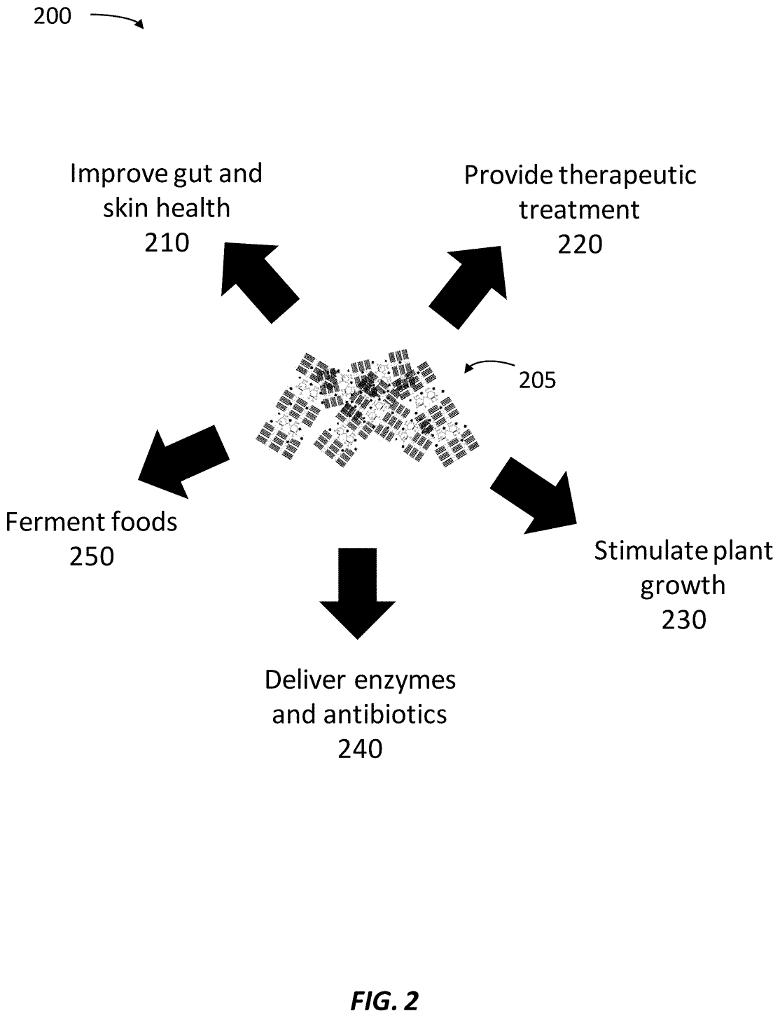

FIG. 2 schematically shows some potential embodiments of applications for vitrified bioactive compositions according to illustrative embodiments. Vitrified bioactive compositions 205 may prepared and packaged to serve many purposes. In some embodiments, vitrified bioactive compositions 205 can be packaged as an oral treatment to improve gut health, or formulated in a medicament to improve skin health 210.

In some embodiments, vitrified bioactive compositions 205 can be packaged as an oral medication to provide therapeutic treatment 220. In particular, as described above, vitrified bioactive compositions 205 can be packaged as enterically-encapsulated capsules, tablets, and/or pellets that can be targeted to deliver therapeutic treatments 220 different regions of the digestive system depending on the type of enteric material incorporated into the coating. Furthermore, the vitrified bioactive compositions 205 may include enzymes and/or antibiotics 240 that me be delivered to the biological subject.

In some embodiments, vitrified bioactive compositions 205 may be used in food processing. For example, vitrified bioactive compositions 205 may be used to aid in the fermentation of foods 250.

In some embodiments, vitrified bioactive compositions 205 may be used to stimulate plant growth 230. In some embodiments, a vitrified bioactive composition 205 may be delivered to a plant, seed, and/or soil by applying powder, and/or pellets, of the vitrified bioactive composition by spraying a solution containing particles and/or powders of enterically encapsulated vitrified bioactive composition 205 on the plant, seed, or soil. In some embodiments, the vitrified bioactive composition 205 may be enterically encapsulated so that the bioactive composition may be protected from spoilage, from being eaten by insects, from exposure to environmental conditions, and the like.

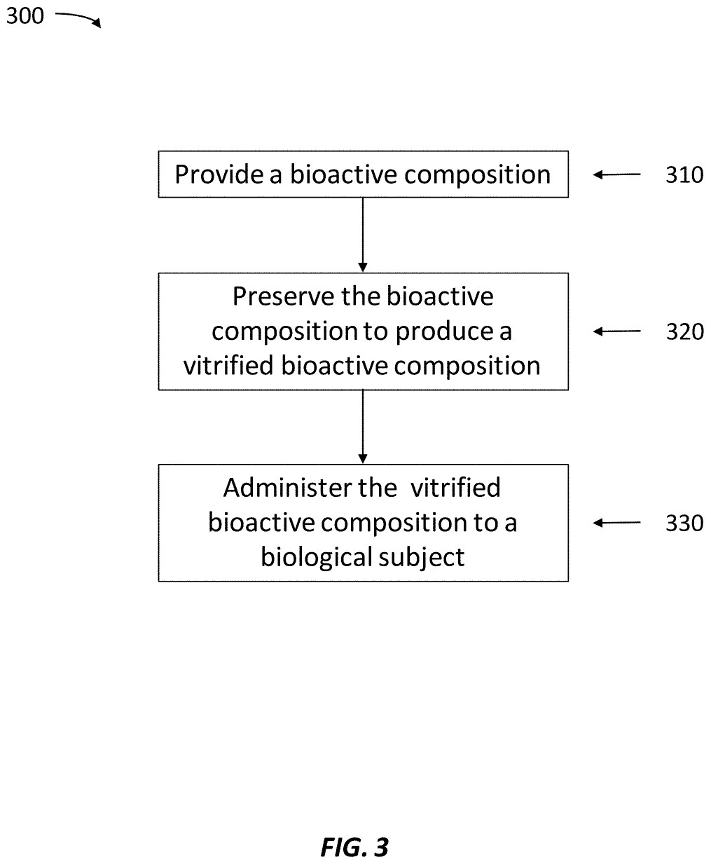

FIG. 3 shows an embodiment of a method 300 of delivery of a bioactive composition in accordance with illustrative embodiments. At 300, a bioactive composition is provided. The bioactive composition may include at least one of microbes, cells, tissue, vaccines, probiotics, antibiotics, vitamins, or mRNA.

At step 320, the bioactive composition is preserved to produce a vitrified bioactive composition. The preservation of the bioactive composition includes vitrification of the bioactive composition in a vitrifying mixture of intrinsically disordered proteins and saccharides to produce a vitrified bioactive composition. The vitrified bioactive composition is configured to release the bioactive composition from the vitrifying mixture.

At step 330, the vitrified bioactive composition is administered to a biological subject. The administration of the vitrified bioactive composition includes applying it to a microbiome of the biological subject.

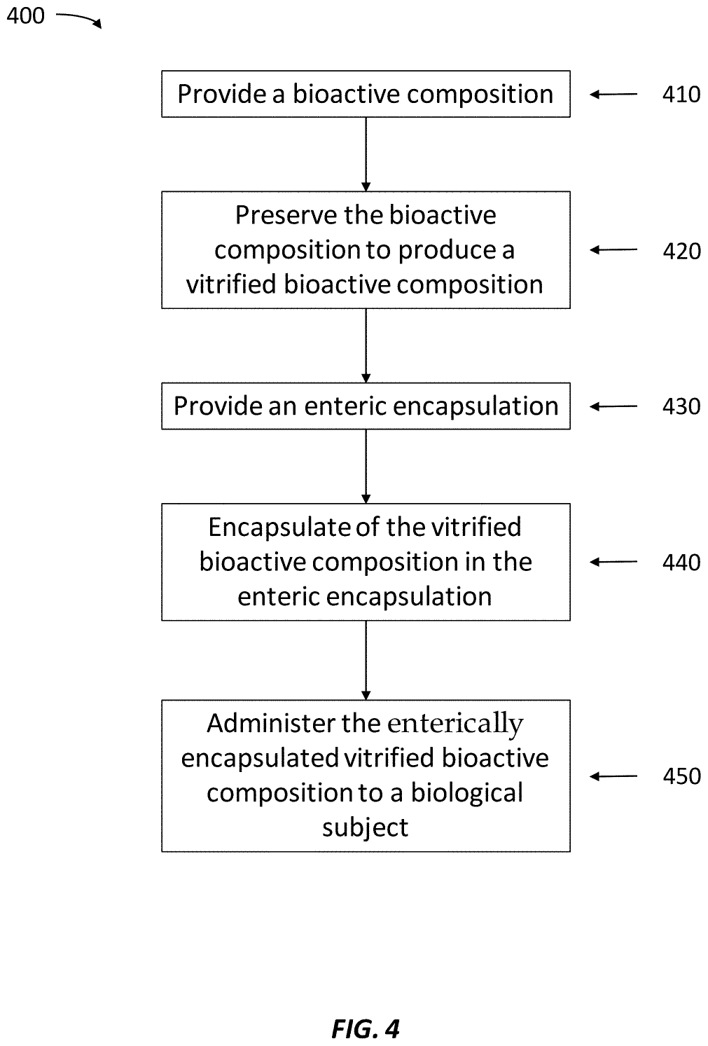

FIG. 4 shows an embodiment of a method 400 of delivery of a bioactive composition in accordance with illustrative embodiments. At 410, a bioactive composition is provided. The bioactive composition includes bioactives, such as at least one of microbes, cells, tissue, vaccines, probiotics, antibiotics, vitamins, or mRNA.

At 420, the bioactive composition is preserved to produce a vitrified bioactive composition. Preserving the bioactive composition includes the vitrification of a vitrifying mixture of intrinsically disordered proteins and saccharides to produce a vitrified bioactive composition. The vitrified bioactive composition is configured to release the bioactive composition from the vitrifying mixture.

At 430, an enteric encapsulation is provided. In some embodiments, the enteric encapsulation may be a capsule that has an enteric polymer coating. In some embodiments, the enteric encapsulation may be a coating that is applied to the vitrified bioactive composition.

At 440, the vitrified bioactive composition is encapsulated in the enteric encapsulation. In some embodiments, the enteric encapsulation encapsulates the vitrified bioactive composition in a capsule comprising an enteric polymer. Alternatively, in some embodiments, the enteric encapsulation coats particles of vitrified bioactive composition with an enteric material.

At 450, the enterically encapsulated vitrified bioactive composition is administered to a biological subject. The administering includes applying the enterically encapsulated vitrified bioactive composition to a microbiome of the biological subject.

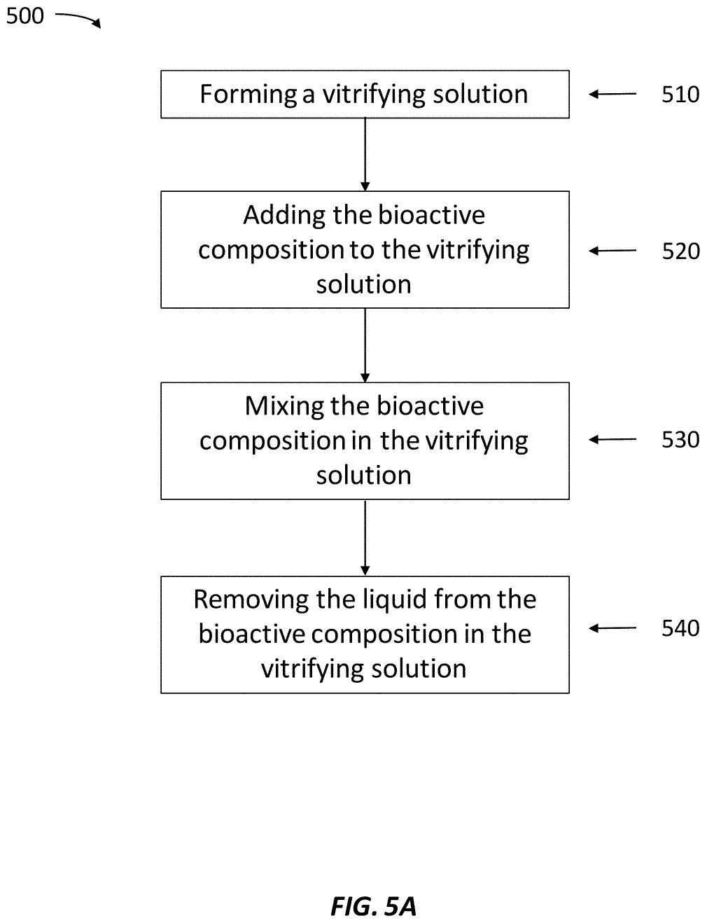

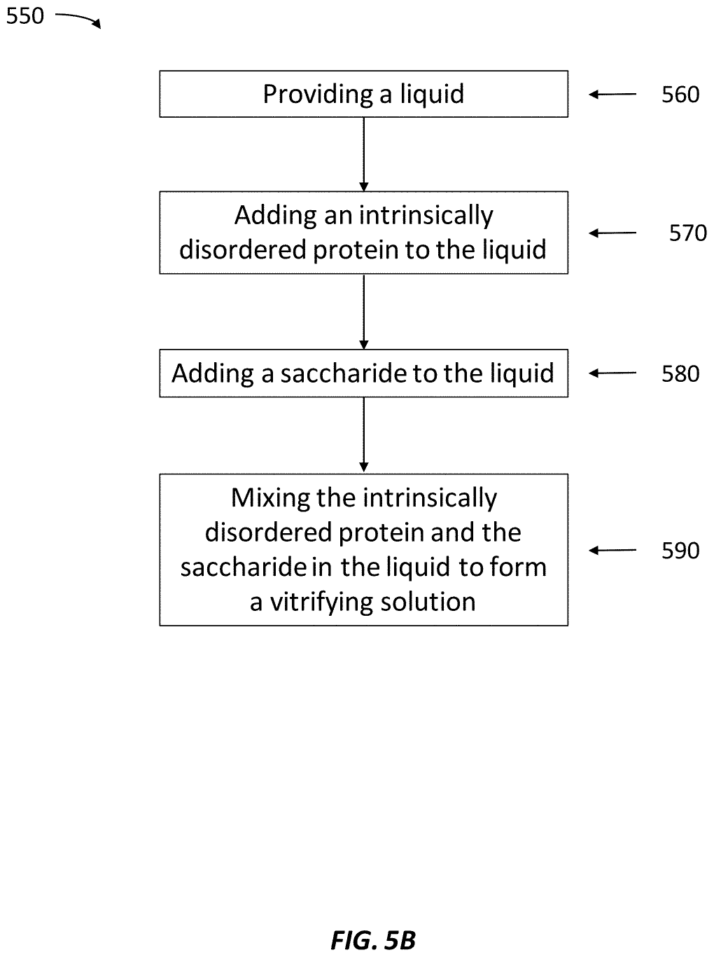

FIG. 5A schematically shows an embodiment of a process to preserve a bioactive composition according to illustrative embodiments. At 510, a vitrifying solution is formed. The vitrifying solution is provided by adding an intrinsically disordered protein and a saccharide to the liquid. The liquid is then mixed.

At 520, the bioactive composition is added to the vitrifying solution. The bioactive composition includes bioactives of at least one of microbes, cells, tissue, vaccines, probiotics, antibiotics, vitamins, or mRNA

At 530, the bioactive composition is mixed in the vitrifying solution. Mixing may be accomplished by a mechanical mixer, a magnetic mixer, manual mixing, and the like.

At 540, the liquid is removed from the bioactive composition in the vitrifying solution. The liquid may be removed by at least one of air-drying, freeze-drying, vacuum drying, or heat-drying.

FIG. 5B schematically shows an embodiment of a process to prepare a vitrifying solution according to illustrative embodiments.

At 560, a liquid is provided. The liquid may be water or an alcohol. in particular, the alcohol may be ethanol.

At 570, an intrinsically disordered protein is added to the liquid. The intrinsically disordered protein may be a naturally occurring protein, or a synthetic polymer.

At 580, a saccharide is added to the liquid. The saccharide may include one or more of polysaccharides, disaccharides, or monosaccharides.

At 590, the intrinsically disordered protein and the saccharide are mixed in the liquid to form a vitrifying solution.

The following examples are intended to further illustrate the disclosure and its preferred embodiments.

### EXAMPLES

### Example 1

Example 1 represents an embodiment where a bioactive microbe composition is applied directly to the outside of a tablet as a film coating. This is done by spray coating tablets with microbes mixed into the IDP/disaccharide mixture. In this example, the premixed microbes and IDP/disaccharide mix are sprayed directly into a gently rotating drum of tablets. After a thorough coating, the tablets continue rotating in the drum until completely dry. At this point, the vitrified bioactive microbe coated tablet is ready for use.

The tablet itself can contain any therapeutic payload, such as vitamins, prebiotics for the microbe, small molecules for disease treatment, biologics, and the like. This vitrified bioactive microbe-coated tablet implementation is most effective for oral delivery of the tablet payload and encapsulated microbes to the gut. After the vitrified bioactive microbes reach the gut, the biodegradable IDP/disaccharide mixture material is dissolved and the microbes are deposited in the lumen of the intestine, where they can deliver their desired therapeutic or probiotic effect.

### Example 2

Example 2 represents an embodiment where a powdered vitrified bioactive composition is formed. A mixture of a bioactive composition is added to a vitrifying solution of silk fibroin, disaccharide, and water to form a solution of a bioactive composition in a vitrifying solution (e.g., vitrifying mixture solution). The vitrifying mixture solution is initially cooled until it freezes at −80 degrees Celsius to form a frozen vitrifying mixture. The frozen vitrifying mixture is then placed in a freeze dryer for about forty-eight hours. The freeze-dryer removes the water frozen vitrifying mixture during this process, and leaves a powder of a vitrified bioactive composition.

This powder of the vitrified bioactive composition can be directly applied to a human being or an animal. The powder can be applied to the skin, to the hair, to the penis, vagina, or foot, to the external auditory canal, to the nostrils, and/or to the mouth.

Alternatively, the powdered vitrified bioactive composition can be dispersed within a lotion or a gel, and then applied to the same regions mentioned above.

In some embodiments, prebiotics and/or microbes may also be mixed with the bioactive composition prior to being mixed into the vitrifying solution.

### Example 3

Example 3 represents an embodiment where powdered vitrified bioactive composition is directly mixed with food or drink and ingested. The ingested vitrified bioactive composition reach microbiomes the mouth, the esophagus, the intestine, and the colon, among others. In this example, the powdered vitrified bioactive composition does not have an enteric coating.

### Example 4

Example 4 represents an embodiment where a powdered vitrified bioactive composition in a tablet form which is coated with an enteric coating. The enterically encapsulated vitrified bioactive composition is ingested, and the vitrified bioactive composition remains intact inside the enteric capsulation until it reaches the intestines. Once in the intestines, the enteric coating breaks down, allowing the vitrified bioactive composition to be broken down and dissolved by the mucous and liquids present in the intestines. The bioactive composition is thereby delivered to the gut microbiome. microbiomes the mouth, the esophagus, the intestine, and the colon, among others.

### Example 5

Example 5 represents an embodiment where a powdered vitrified bioactive composition is mixed with a filler (and, optionally, prebiotics) and pressed into a tablet form. In this example, the powdered vitrified bioactive composition pressed into a pellet with the filler does not have an enteric coating. However, the filler together with vitrifying composition provide the protection of the bioactive composition in acidic stomach acid and basic bile, as well as proteases in the gut. In this embodiment, the tablet of the vitrified bioactive composition mixed with a filler is able to reach the lower gastrointestinal tract, influencing the microbiome there.

### Example 6

Example 6 represents an embodiment where microbes remain in a liquid or gel solution of IDP and disaccharide. In embodiments, the gel solution is made of a gel-forming material, such as gelatin (made of collagen), and loaded with prebiotics to support microbe growth. The microbes in their liquid or gel vitrifying solution are be loaded into an enterically-coated capsule (e.g., pH-activated capsule). The capsule will break apart once it passes through the stomach acid and bile, releasing its payload of mucus-adherent hydrogel harboring encapsulated commensal microbes.

### Example 7

Example 7 represents illustrative embodiments that enhance the health of the human skin microbiome and control the release of microbes and other bioactive components in a sustainable and effective manner. Among other places, illustrative embodiments can be applied in the following areas: the skin, nostril, mouth, vagina, hair, foot, and penis.

In some embodiments, the freeze-dried vitreous bioactive composition mixture can then be applied as a powder or reconstituted with the addition of water or a polymer into a solution that is applied to a biological subject. The film can be applied to facial masks or swallowed for delivery in the mouth and the gel can be applied on your skin or applied in your mouth. The solution can be applied as a spray for the skin or mouth or nasal cavity.

In some embodiments, a mixture of intrinsically disordered proteins (IDPs) with a disaccharide are mixed with one or both live or dead microbes. The mixture is enhanced with the addition of some metabolites or preservatives. The mixture is then air-dried into a film that can be water annealed, or ethanol-annealed to promote solidification of the silk fibroin and saccharides in the complex. The film can then be ingested to deliver microbes in the mouth. The film may also be applied to a facial mask for delivery and added to band aids for injuries.

In some embodiments, a solution containing a vitrified bioactive composition comprising microbes and other bioactive components is formed. The solution may have preservatives or metabolites added to the solution to enhance performance. The solution may be applied as a spray on a subject's skin, hair, nostril, vagina, foot, and/or penis. The solution may be kept in an airtight container. The solution may also be used as a nasal spray and thereby applied to the nostrils, esophagus, and lungs.

The embodiments of the invention described above are intended to be merely exemplary; numerous variations and modifications will be apparent to those skilled in the art. Such variations and modifications are intended to be within the scope of the present invention as defined by any of the appended claims.

What is claimed is:

1. A method of delivery of a bioactive composition to a biological subject, comprising:
providing a bioactive composition, the bioactive composition comprising bioactives of at least one of microbes, cells, tissue, vaccines, probiotics, antibiotics, vitamins, peptides, RNAi, or mRNA;
preserving the bioactive composition, the preserving comprising vitrification of the bioactive composition in a vitrifying mixture of intrinsically disordered proteins and saccharides to produce a vitrified bioactive composition, the vitrified bioactive composition is configured to release the bioactive composition from the vitrifying mixture; and
administering the vitrified bioactive composition to the biological subject, the administering comprises applying the vitrified bioactive composition to a microbiome of the biological subject.

2. The method of claim 1, further comprising:
providing an enteric encapsulation of the vitrified bioactive composition, wherein:
the enteric encapsulation is configured to protect the vitrified bioactive composition from a first set of environmental conditions; and
the enteric encapsulation is configured to breakdown in a second set of environmental conditions, thereby releasing the vitrified bioactive composition.

3. The method of claim 2, wherein the enteric encapsulation comprises:
an enteric polymer capsule in which the vitrified bioactive composition is placed; or
an enteric coating that entirely covers the vitrified bioactive composition.

4. The method of claim 1, wherein:
the intrinsically disordered proteins comprise at least one of a tardigrade intrinsically disordered protein, a silk fibroin, or tropoelastin; and
the saccharides are a disaccharide.

5. The method of claim 1, wherein the microbes comprise at least one of a fungus, a eubacterium, a eukaryotic organism, or an archaebacteria.

6. The method of claim 1, wherein the bioactive composition is one of: anaerobic, aerobic, spore-forming, vegetative, native to a human microbiome, native to an animal microbiome, non-native to the human microbiome, non-native to the animal microbiome, native to a plant microbiome, non-native to the plant microbiome, an engineered version of the bioactive composition that is non-native to the human microbiome, or an engineered version of the bioactive composition that is non-native to the animal microbiome.

7. The method of claim 1, wherein the bioactive composition is enhanced by an addition of metabolites and/or preservatives.

8. The method of claim 1, wherein the biological subject is a human being.

9. The method of claim 8, wherein the applying comprises applying the vitrified bioactive composition to at least one of skin, a vagina, a penis, a foot, or an armpit of the human being.

10. The method of claim 1, wherein the applying comprises ingesting an enterically encapsulated vitrified bioactive composition in a form of at least one of an oral ingestible pill, an oral ingestible tablet, or a solution.

11. The method of claim 10, wherein:
the enterically encapsulated vitrified bioactive composition is ingested in the form of an enterically encapsulated vitrified bioactive composition that enters a digestive track of the biological subject;
the enterically encapsulated vitrified bioactive composition passes through the digestive track intact until the enterically encapsulated vitrified bioactive composition experiences a pH of greater than about 6; and
the enterically encapsulated vitrified bioactive composition breaks down and releases the vitrified bioactive composition when it experiences the pH of greater than about 6; and
the vitrified bioactive composition releases the bioactive composition.

12. The method of claim 1, wherein the vitrifying the bioactive composition comprises lowering the metabolism of the bioactives of the bioactive composition, thereby putting the bioactives in stasis.

13. The method of claim 1, wherein the vitrifying the bioactive composition protects bioactive composition from dying due to exposure from oxygen.

14. The method of claim 10, wherein;
the vitrified bioactive composition is released from the enterically encapsulated vitrified bioactive composition in a gut of the biological subject when the enterically encapsulated vitrified bioactive composition experiences a pH greater than a pH of about 6;
the vitrified bioactive composition is not released from the enterically encapsulated vitrified bioactive composition in a stomach of the biological subject, because a pH of the stomach is less than a pH of about 5.

15. The method of claim 14, wherein:
the vitrified bioactive composition that is released in the gut of the biological subject adheres to intestinal mucus, rehydrates, resuscitates, and proliferates; and
the bioactive composition is released over a duration from the vitrified bioactive composition.

16. The method of claim 15, wherein the bioactive composition that is released in the gut of the biological subject releases at least one of viable and healthy microbes, cells, tissue, vaccines, probiotics, antibiotics, or mRNA.

17. The method of claim 4, wherein:
the silk fibroin is extracted from Bombyx Mori; and
the disaccharide is trehalose.

18. The method of claim 1, wherein prebiotics are included in the bioactive composition to provide energy to nourish the bioactives.

19. The method of claim 1, wherein:
the biological subject is a plant, a seed, or a soil;
the applying comprises spraying a solution containing particles and/or a powder of enterically encapsulated vitrified bioactive composition on the plant, seed, or soil.

20. A composition for application to a microbiome of a biological subject, comprising:
a bioactive composition, the bioactive composition comprising bioactives of at least one of microbes, cells, tissue, vaccines, probiotics, antibiotics, vitamins, or mRNA; and
a vitrifying mixture, the vitrifying mixture comprising intrinsically disordered proteins and saccharides, wherein the bioactive composition is preserved in the vitrifying mixture to form a vitrified bioactive composition.

21. The composition of claim 20, wherein the composition further comprises:
an enteric encapsulant, the enteric encapsulant configured to encapsulate the vitrified bioactive composition to protect the vitrified bioactive composition from a first set of environmental conditions and to release the vitrified bioactive composition in a second set of environmental conditions.

22. The composition of claim 20, wherein the bioactive is freeze dried in preparation for being preserved in the vitrifying mixture.

23. The composition of claim 20, wherein the bioactive composition further comprises a probiotic and/or an antibiotic.

24. The composition of claim 20, wherein the vitrified bioactive composition is incorporated in a skin care lotion.

25. The composition of claim 20, wherein the vitrified bioactive composition is incorporated in a wound healing medication.

26. The composition of claim 20, wherein:
the intrinsically disordered proteins are a silk fibroin; and
the saccharides are a disaccharide.

27. The composition of claim 20, wherein the intrinsically disordered proteins comprise:
a naturally occurring protein; or
a synthetic polymer.

28. A method of delivery of a bioactive composition to a biological subject, comprising:
providing a bioactive composition, the bioactive composition comprising at least one bioactive;
preserving the bioactive composition, the preserving comprising vitrification of the bioactive composition in a vitrifying mixture of intrinsically disordered proteins and saccharides to produce a vitrified bioactive composition, the vitrified bioactive composition is configured to release the bioactive composition from the vitrifying mixture;
providing an enteric encapsulation;
encapsulating the vitrified bioactive composition in the enteric encapsulation; and
administering the enterically encapsulated vitrified bioactive composition to the biological subject, the administering comprises applying the enterically encapsulated vitrified bioactive composition to a microbiome of the biological subject.

29. The method of claim 28, wherein the enteric encapsulation comprises either:
encapsulating the vitrified bioactive composition in a capsule comprising an enteric polymer; or
a coating particles of vitrified bioactive composition with an enteric material.

30. The method of claim 28, wherein the bioactives comprise at least one of microbes, cells, tissue, vaccines, probiotics, antibiotics, vitamins, or mRNA.

31. The method of claim 28, wherein:
the intrinsically disordered proteins are a silk fibroin; and
the saccharides are a disaccharide.

32. A method of preserving a bioactive composition, comprising:
forming a vitrifying solution, comprising:
providing a liquid,
adding an intrinsically disordered protein to the liquid;
adding a saccharide to the liquid;
mixing the intrinsically disordered protein and the saccharide in the liquid to form the vitrifying solution;
adding the bioactive composition to the vitrifying solution;
mixing the bioactive composition in the vitrifying solution; and
removing the liquid from the bioactive composition in the vitrifying solution.

33. The method of claim 32, further comprising:
heating the bioactive composition in the vitrifying solution to a temperature of between 20 degrees Celsius and 125 degrees Celsius.

34. The method of claim 32, further comprising:
cooling the bioactive composition in the vitrifying solution to a temperature of between 0 degrees Celsius and −80 degrees Celsius.

34. The method of claim 32, wherein removing the liquid comprises at least one of air-drying, freeze-drying, vacuum drying, or heat-drying.

35. The method of claim 32, wherein the liquid comprises water or an alcohol.
